(19) United States
(12) Patent Application Publication
(10) Pub. No.: US 2024/0293019 A1
(43) Pub. Date: Sep. 5, 2024

(54) PORTABLE MEDICAL DEVICE WITH TONGUE DEPRESSOR APPARATUS

(71) Applicant: GLOBALMEDIA GROUP, LLC, Scottsdale, AZ (US)

(72) Inventors: Michael D. Harris, Scottsdale, AZ (US); Jeremy Troutman, Scottsdale, AZ (US)

(73) Assignee: GLOBALMEDIA GROUP, LLC, Scottsdale, AZ (US)

(21) Appl. No.: 18/657,520

(22) Filed: May 7, 2024

Related U.S. Application Data
(63) Continuation-in-part of application No. 18/117,340, filed on Mar. 3, 2023.

(51) Int. Cl. A61B 1/24; A61B 1/04; A61B 13/00

(52) U.S. Cl. CPC A61B 1/24; A61B 1/042; A61B 13/00

(57) ABSTRACT

An oral examination camera system that allows a user to conduct an oral examination by depressing an individual's tongue while simultaneously recording the examination. A disposable tongue depressor inserted into a tongue depressor apparatus while the tongue depressor apparatus is coupled to a portable medical device. The portable medical device being configured to transmit video data from at least one camera to the telemedicine management system. The telemedicine management system can be configured to share the video data through a videoconference.

### CROSS-REFERENCE TO RELATED APPLICATIONS

This is a continuation in part of U.S. application Ser. No. 18/117,340, filed Mar. 3, 2023 and entitled “PORTABLE MEDICAL DEVICES, SYSTEMS, AND METHODS OF OPERATION.” The foregoing application is hereby incorporated in its entirety by reference for all purposes.

### FIELD

The present disclosure generally relates to telemedicine devices, systems, and methods, and more particularly to a portable medical device with a tongue depressor apparatus and methods of using the tongue depressor apparatus.

### BACKGROUND

Telemedicine, or telehealth, allows a health care provider to care for a patient without an in-person office visit. Telemedicine is primarily performed online with access on a computer, tablet, or smartphone. For example, telemedicine can include a phone call, a video chat, texting between patients and doctors, and/or remote monitoring (i.e., a patient taking his or her temperature and/or blood pressure) and providing the measurements to a doctor.

Virtual visits are growing in popularity. Telehealth can provide various benefits, such as reduced exposure of sick patients to the public, reduced travel time for patients, shorter wait times for appointments, and/or increased access to specialists that are located far away from a patient's residence. Accordingly, portable medical devices, systems, and methods of operation that facilitate telemedicine may be desirable.

### SUMMARY

Disclosed herein is a tongue depressor apparatus that is configured for home use and/or use in emergency situations, such as on an airplane, on a ship, or the like. In various embodiments, the tongue depressor apparatus can help facilitate an in-home checkup exam of a patient, without the patient having to visit a respective doctor's office. In various embodiments, the tongue depressor apparatus includes a main body configured to attach to a portable medical device to help facilitate the examination of a throat of a patient.

A tongue depressor apparatus is disclosed herein. In various embodiments, the tongue depressor apparatus comprises a main body extending from a first end to a second end; a flange disposed at the first end.

In various embodiments, the main body defines a longitudinal axis, the flange is substantially parallel to the longitudinal axis.

In various embodiments, the tongue depressor apparatus main body has an aperture disposed at a first end, extending from the first end of the tongue depressor apparatus main body to the second end of the tongue depressor apparatus main body.

In various embodiments, the aperture is configured to detachably couple to a portable medical device.

In various embodiments, the tongue depressor apparatus further comprises a slot extending from the first end to the second end of the tongue depressor apparatus main body.

In various embodiments, the slot is configured to hold a disposable tongue depressor.

An oral examination camera assembly is disclosed herein. In various embodiments, the oral examination camera assembly comprises: a portable medical device including a portable medical device main body, a camera, the portable medical device main body extending from a first end to a second end, the camera disposed at the first end; a tongue depressor apparatus configured to detachably couple to the portable medical device main body.

In various embodiments, the camera defines a first optical axis and the portable medical device main body defines a longitudinal axis, wherein the first optical axis and the longitudinal axis are substantially parallel.

In various embodiments, the portable medical device main body of the tongue depressor apparatus defines a longitudinal axis, wherein the portable medical device main body and the tongue depressor apparatus main body are substantially parallel.

A method is disclosed herein. In various embodiments, the method comprises removing the oral examination camera system from a user's clothing and inserting a disposable tongue depressor into the tongue depressor apparatus. In various embodiments, the method comprises using the tongue depressor apparatus in conjunction with the portable medical device to depress a tongue of a patient and visually record a portion of an oral examination of the patient simultaneously with the depressing of the tongue of the patient; removing and discarding the disposable tongue depressor from the oral examination camera system after the oral examination of the patient is complete.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the following detailed description and claims in connection with the following drawings. While the drawings illustrate various embodiments employing the principles described herein, the drawings do not limit the scope of the claims.

FIG. 1 illustrates a schematic view of a telemedicine system, in accordance with various embodiments.

FIG. 2A illustrates a perspective view of a portable medical device for use in a telemedicine system, in accordance with various embodiments.

FIG. 2B illustrates a cross-sectional view of the portable medical device of FIG. 2A, in accordance with various embodiments.

FIG. 2C illustrates a perspective view of a portable medical kit having a portable medical device, in accordance with various embodiments.

FIG. 3 illustrates a top perspective view of a tongue depressor apparatus, in accordance with various embodiments.

FIG. 4 illustrates a bottom perspective view of a tongue depressor apparatus, in accordance with various embodiments.

FIG. 5 illustrates a perspective view of an oral examination camera system, in accordance with various embodiments.

FIG. 6 illustrates a perspective view of a tongue depressor apparatus with a disposable tongue depressor inserted into the tongue depressor apparatus, in accordance with various embodiments.

FIG. 7 illustrates a perspective view of a tongue depressor apparatus detachably coupled to a portable medical device, in accordance with various embodiments.

FIG. 8 illustrates a flowchart with steps for a method of using an oral examination camera system, in accordance with various embodiments.

### DETAILED DESCRIPTION

The detailed description of various embodiments herein makes reference to the accompanying drawings and pictures, which show various embodiments by way of illustration. While these various embodiments are described in sufficient detail to enable those skilled in the art to practice the disclosure, it should be understood that other embodiments may be realized and that logical and mechanical changes may be made without departing from the spirit and scope of the disclosure. Thus, the detailed description herein is presented for purposes of illustration only and not for purposes of limitation. For example, the steps recited in any of the method or process descriptions may be executed in any order and are not limited to the order presented. Moreover, any of the functions or steps may be outsourced to or performed by one or more third parties. Modifications, additions, or omissions may be made to the systems, apparatuses, and methods described herein without departing from the scope of the disclosure. For example, the components of the systems and apparatuses may be integrated or separated. An individual component may be comprised of two or more smaller components that may provide a similar functionality as the individual component. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set. Furthermore, any reference to singular includes plural embodiments, and any reference to more than one component may include a singular embodiment. Although specific advantages have been enumerated herein, various embodiments may include some, none, or all of the enumerated advantages.

Systems, methods, and computer program products are provided. In the detailed description herein, references to “various embodiments,”“one embodiment,”“an embodiment,”“an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

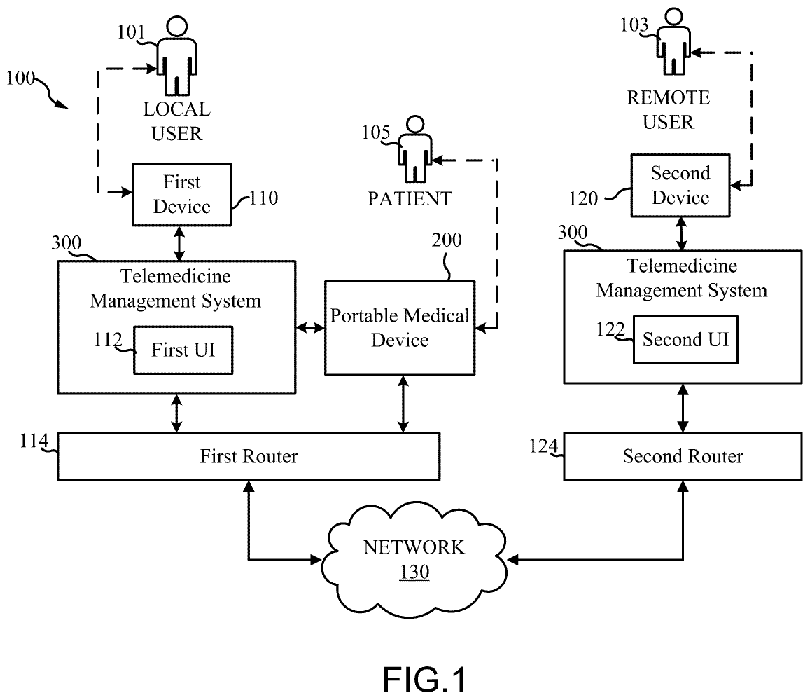

Referring now to FIG. 1, a schematic view of a telemedicine system 100 for facilitating a remote diagnosis and treatment of a patient 105 with a portable medical device 200, in accordance with various embodiments. In various embodiments, the telemedicine system 100 can be configured to facilitate a home visit (e.g., by a parent, sibling, guardian, the patient, etc.). In various embodiments, the telemedicine system 100 can be configured to facilitate communications with a doctor in an emergency situation (e.g., on an airplane, a boat, or the like). The present disclosure is not limited in this regard.

As shown in FIG. 1, the telemedicine system 100 comprises a telemedicine management system 300. As will be appreciated by one of ordinary skill in the art, the telemedicine management system 300 may be embodied as a customization of an existing system, an add-on product, a processing apparatus executing upgraded software, a stand-alone system, a distributed system, a method, a data processing system, a device for data processing, and/or a computer program product. Accordingly, any portion of the system or a module may take the form of a processing apparatus executing code, an internet-based embodiment, an entirely hardware embodiment, or an embodiment combining aspects of the internet, software, and hardware. Furthermore, the system may take the form of a computer program product on a computer-readable storage medium having computer-readable program code means embodied in the storage medium. Any suitable computer-readable storage medium may be utilized, including hard disks, optical storage devices, magnetic storage devices, and/or the like.

In various embodiments, the telemedicine management system 300 comprises a computer-based system. In various embodiments, components, modules, and/or engines of the telemedicine management system 300 may be implemented as micro-applications or micro-apps. Micro-apps are typically deployed in the context of a mobile operating system, including for example, a WINDOWS® mobile operating system, an ANDROID® operating system, an APPLE® iOS operating system, a BLACKBERRY® company's operating system, and the like. The micro-app may be configured to leverage the resources of the larger operating system and associated hardware via a set of predetermined rules which govern the operations of various operating systems and hardware resources. For example, where a micro-app desires to communicate with a device or network other than the mobile device or mobile operating system, the micro-app may leverage the communication protocol of the operating system and associated device hardware under the predetermined rules of the mobile operating system. Moreover, where the micro-app desires an input from a user, the micro-app may be configured to request a response from the operating system which monitors various hardware components and then communicates a detected input from the hardware to the micro-app.

In various embodiments, the telemedicine management system 300 may include a software program stored in a memory of a computer, accessible through the Internet, or the like. In various embodiments, the software program of the telemedicine management system 300 is configured to perform the methods described herein in a memory (e.g., a local memory, a cloud-based memory, or the like) and run the software program using a processor of the first device 110 (e.g., a computer) and/or the second device 120 (e.g., a computer). The first device 110 and the second device 120 may each include any number of individual processors and memories. Various data (e.g., video data, sound data, etc.) may be communicated between the first device 110 of a local user 101 (e.g., a parent, a guardian, a patient 105, a local doctor, a local nurse, etc.) via the first UI 112 of the telemedicine management system 300, the portable medical device 200, and the second device 120 of the remote user 103 (e.g., a remote doctor, a remote nurse, etc.) via the second UI 122 of the telemedicine management system 300 as described further herein. For example, the first device 110 can be in electronic communication with the second device 120 through a network 130. Such information may also be communicated between the external devices (e.g., first device 110, and/or second device 120) through the telemedicine management system 300 by use of the network 130 (e.g., through any network 130 such as a local area network (LAN), or wide area network (WAN) such as the Internet).

As used herein, the term “network” includes any cloud, cloud computing system, or electronic communications system or method which incorporates hardware and/or software components. Communication among the parties may be accomplished through any suitable communication channels, such as, for example, a telephone network, an extranet, an intranet, internet, point of interaction device (point of sale device, personal digital assistant (e.g., an IPHONE® device, a BLACKBERRY® device), cellular phone, kiosk, etc.), online communications, satellite communications, off-line communications, wireless communications, transponder communications, local area network (LAN), wide area network (WAN), virtual private network (VPN), networked or linked devices, keyboard, mouse, and/or any suitable communication or data input modality. Moreover, although the system is frequently described herein as being implemented with TCP/IP communications protocols, the system may also be implemented using IPX, APPLETALK® program, IP-6, NetBIOS, OSI, any tunneling protocol (e.g., IPsec, SSH, etc.), or any number of existing or future protocols. If the network is in the nature of a public network, such as the internet, it may be advantageous to presume the network to be insecure and open to eavesdroppers. Specific information related to the protocols, standards, and application software utilized in connection with the internet may be contemplated.

“Cloud” or “Cloud computing” includes a model for enabling convenient, on-demand network access to a shared pool of configurable computing resources (e.g., networks, servers, storage, applications, and services) that can be rapidly provisioned and released with minimal management effort or service provider interaction. Cloud computing may include location-independent computing, whereby shared servers provide resources, software, and data to computers and other devices on demand.

As used herein, “transmit” may include sending electronic data from one system component to another over a network connection. Additionally, as used herein, “data” may include encompassing information such as commands, queries, files, data for storage, and the like in digital or any other form.

The telemedicine management system 300 may receive and display information (such as video data, selection devices to select cameras, etc.) via the first UI 112 and/or the second UI 122. The user interfaces (e.g., the first UI 112 and/or the second UI 122) may include various peripheral output devices (such as monitors and printers), as well as any suitable input or control devices (such as a mouse and keyboard) to allow users to control and interact with the software program.

In various embodiments, first device 110 and second device 120 may each be in electronic communication with the telemedicine management system 300, directly or via a respective user interface (e.g., first UI 112 and/or second UI 122). First device 110 and second device 120 may comprise any suitable hardware, software, and/or database components capable of sending, receiving, and storing data. For example, first device 110 and/or second device 120 may comprise a personal computer, personal digital assistant, cellular phone, smartphone (e.g., IPHONE®, BLACKBERRY®, and/or the like), IoT device, telehealth device, and/or the like. First device 110 and/or second device 120 may comprise an operating system, such as, for example, a WINDOWS® mobile operating system, an ANDROID® operating system, APPLE® IOS®, a BLACKBERRY® operating system, a LINUX® operating system, and the like. First device 110 and/or second device 120 may also comprise software components installed on first device 110 and/or second device 120 and configured to enable access to various telemedicine management systems 300 components. For example, first device 110 and/or second device 120 may comprise a web browser (e.g., MICROSOFT INTERNET EXPLORER®, GOOGLE CHROME®, etc.), an application, a micro-app or mobile application, or the like, configured to allow first device 110 and/or second device 120 to access and interact with the telemedicine management system 300 (e.g., directly or via a respective UI, as discussed further herein).

In various embodiments, first device 110 may be configured to communicate with and/or interact with telemedicine management system 300 via first UI 112.

First UI 112 may comprise a graphical user interface (GUI) accessible via a mobile application, web browser, software application, or the like. For example, first device 110 may interact with first UI 112 to instruct the telemedicine management system 300 to electronically couple the portable medical device 200 to the telemedicine management system 300 (e.g., through a first router 114 or directly). In this regard, as described further herein, video data from a camera of the portable medical device 200 can be transmitted from the portable medical device 200 to the first device 110 through the telemedicine management system 300. Further, as will be discussed further herein, the video data from the portable medical device 200 can be shared by the local user 101 through the first UI 112 of the telemedicine management system 300 across the network 130 to remote user 103 through the second UI 122 of the telemedicine management system 300 displayed on the second device 120, which can be connected to the network 130 through second router 124. Accordingly, the telemedicine system 100 and the telemedicine management system 300 disclosed herein are configured to transmit video data from a portable medical device 200 (e.g., video data of a patient 105) from a first location (e.g., a location of the local user 101 and the patient 105) to a second location (i.e., a remote location of a remote user 103).

In various embodiments, the telemedicine management system 300 disclosed herein is configured to facilitate a telehealth visit where basic examinations (i.e., such as those typically performed in a doctor's office) can be performed. For example, as will be discussed in more detail further herein, the portable medical device 200 can be configured with an otoscope to perform an ear inspection of the patient 105 and/or a general camera with a fixed magnification configured to record (or stream) video data from a general examination (e.g., of the throat or nose of the patient 105).

In various embodiments, the portable medical device 200 can be operated by the patient 105 directly or a user (e.g., a parent, a guardian, a significant other, a sibling, nurse, etc.). Similarly, either the local user 101 or the patient 105 can operate the first UI 112 of the telemedicine management system 300 to ensure communication with the remote user 103 as described further herein.

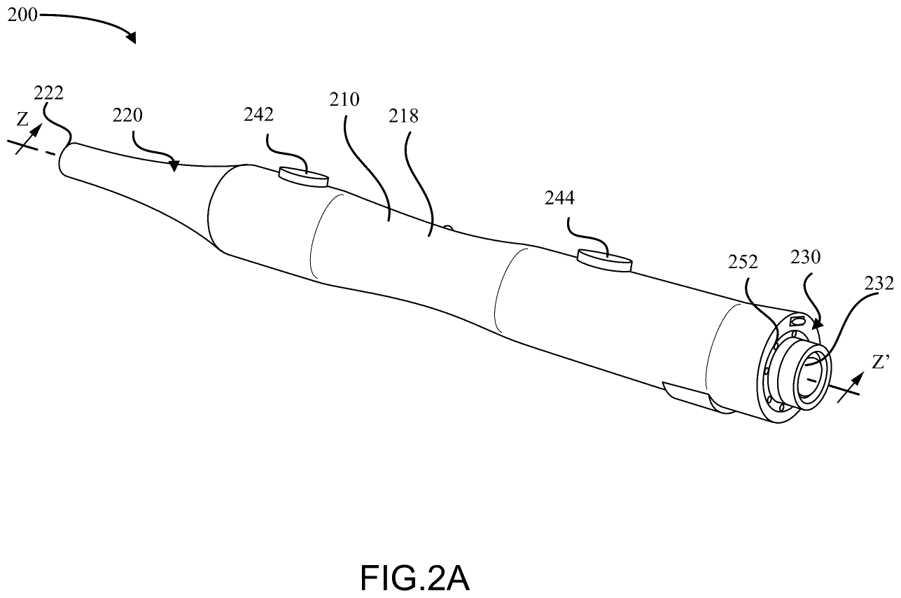

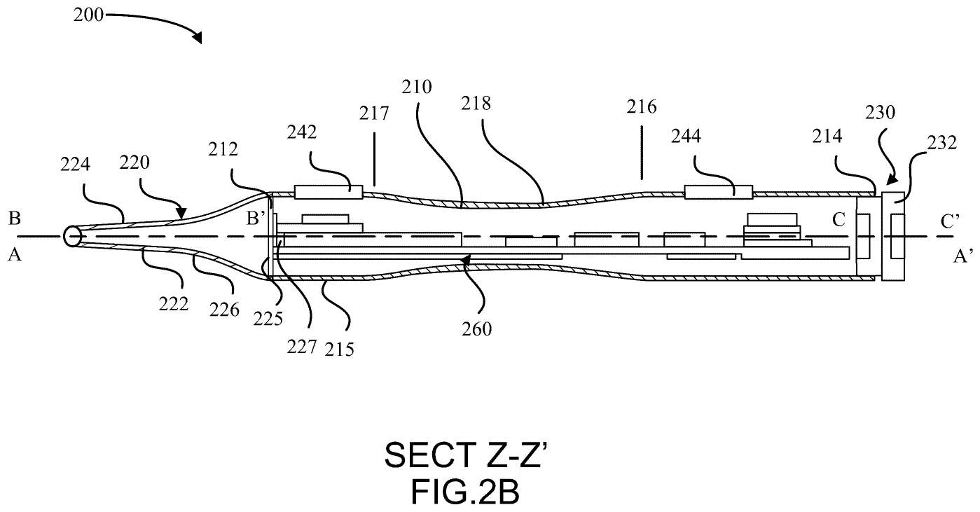

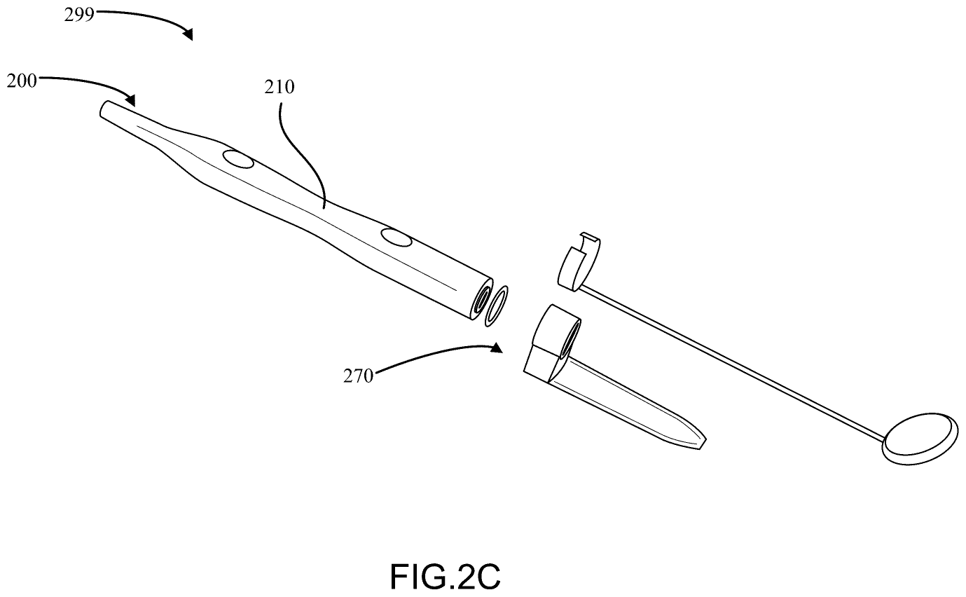

Referring now to FIGS. 2A and 2B, a perspective view of a portable medical device 200 (FIG. 2A) and a cross-sectional view of the portable medical device 200 along section line Z-Z′ (FIG. 2B) for use in a telemedicine system are illustrated, in accordance with various embodiments. The portable medical device 200 is configured for home use (e.g., by a parent, sibling, guardian, etc.), for use in an emergency situation (e.g., on an airplane, on a boat, etc.), or the like. The present disclosure is not limited in this regard. In various embodiments, the portable medical device 200 is configured to perform various functions associated with a telemedicine system.

The portable medical device 200 comprises a main body 210 extending longitudinally from a first end 212 to a second end 214, a camera 220 disposed at the first end 212, and a camera 230 disposed at the second end 214. Although illustrated with dual cameras (i.e., cameras 220, 230), the present disclosure is not limited in this regard. For example, a medical device having only a single camera (e.g., only camera 220 or only camera 230) is within the scope of this disclosure.

The camera 220 can be configured with otoscopic lensing (i.e., lensing designed for inspecting an ear of a patient), and the camera 230 can be configured with general lensing (i.e., a general video scope configured for throat inspections, nose inspections, or other general inspections). Accordingly, the portable medical device 200 can be configured for both general medical exams and otoscopic inspections and general inspections, in accordance with various embodiments.

In various embodiments, the main body 210 defines a longitudinal axis A-A′. For example, the main body 210 can define a centerline that corresponds to the longitudinal axis A-A′. Similarly, the camera 220 defines an optical axis B-B′, and the camera 230 defines an optical axis C-C′. In various embodiments, the optical axis B-B′ of the camera 220 and the optical axis C-C′ are substantially parallel to the longitudinal axis A-A′ of the main body 210.

“Substantially parallel” as referred to herein, is parallel +/−10 degrees or +/−5 degrees. In various embodiments, the optical axis B-B′ of the camera 220 and the optical axis C-C′ of the camera 230 are co-axial to each other. In various embodiments, the optical axis B-B′ of the camera 220 and the optical axis C-C′ of the camera 230 are co-axial with the longitudinal axis A-A′. In various embodiments, by having parallel and/or co-axial cameras 220, 230, the portable medical device 200 can be easier to manufacture relative to having optical axis which are non-parallel. In various embodiments, by having cameras 220, 230 parallel with the longitudinal axis A-A′, the optical axis can be easily aligned by a user during use (e.g., by referencing a direction of the portable medical device 200).

In various embodiments, the main body 210 defines a grip 218. For example, the main body 210 can comprise a varying cross-sectional diameter as the main body 210 that extends from the first end 212 to the second end 214. In various embodiments, the grip 218 can be defined between a first axial location 216 and a second axial location 217. Between the first axial location 216 and the second axial location 217, the cross-sectional diameter of the main body 210 can decrease from a first diameter at the first axial location 216 to a minimum diameter (e.g., proximal to a midpoint axially along the grip 218) and increase to second diameter at the second axial location 217. In this regard, the grip 218 can be configured to receive a hand to hold the portable medical device 200 in an ergonomic fashion.

In various embodiments, the main body 210 can comprise a membrane 215. For example, the membrane 215 can be thin and tactile (e.g., between 0.01 inches (0.025 cm) and 0.25 inches (0.635 cm), or between 0.01 inches (0.025 cm) and 0.225 inches (0.32 cm), or the like). In various embodiments, the membrane 215 can be an elastomeric material. In this regard, the membrane 215 can be configured to cover harder components, such as a printed circuit board, a housing, and/or various internal components of the portable medical device 200 disposed within the main body 210. In various embodiments the membrane 215 is configured to provide a soft exterior for gripping for an ergonomic benefit to users, in accordance with various embodiments.

In various embodiments, the camera 220 comprises a lens 222. The lens 222 can include a fixed magnification. For example, the lens 222 can comprise a lens having a fixed magnification between 15× and 20×, or approximately 18×. In various embodiments, the lens 222 is distinct from the camera 220. In various embodiments, the lens 222 is an otoscopic lens 224. For example, the otoscopic lens 224 can comprise a magnifying lens 225, a speculum 226, and a light source 227. The speculum 226 of the otoscopic lens 224 can be detachable and include an insert configured to press fit into an aperture at the first end 212 of the main body 210. In this regard, the speculum 226 of the otoscopic lens 224 can be coupled to the lens to the main body 210 in an easy and straightforward manner, in accordance with various embodiments. Although described as being detachable, the speculum 226 is not limited in this regard. For example, the speculum 226 of the otoscopic lens 224 can be integral (i.e., form one monolithic piece) with the main body 210 and be within the scope of this disclosure.

In various embodiments, the camera 230 comprises a video scope 232 configured for adjustable magnification of an object. For example, video scope 232 of the camera 230 can comprise a 2×-4× near-far general video scope, a 0.5×-2× near-far general video scope, or the like. The present disclosure is not limited in this regard.

In various embodiments, the portable medical device 200 further comprises a button 242 and a button 244. The button 242 is electronically (e.g., wired or wirelessly) coupled to the camera 220. Similarly, the button 244 is electronically coupled the camera 230. In various embodiments, the grip 218 is disposed axially between the button 242 and the button 244. In this regard, a thumb of a user can be oriented towards a button that controls a camera that the user is using. For example, in response to using the camera 220, a field of view of the camera 220 will be directed in an axial direction away from the main body 210 of the portable medical device 200. Accordingly, a user's hand will be around the grip 218 and a thumb of the user will be oriented towards button 242, which controls the camera 220. Similarly, in response to using camera 230, a field of view of the camera 230 will be directed in an axially opposite direction from the camera 220. In this regard, a user's hand will be oriented in an opposite direction from the axial direction when the camera 220 is in use. Accordingly, the thumb of the user's hand will be oriented towards the button 244, which controls the camera 230.

In various embodiments, the button 242 is configured to freeze a frame of the camera 220 in response to being depressed. For example, when video data from the camera 220 is being fed to a doctor (e.g., via a telemedicine system as described further herein), the doctor may want the patient to pause the camera 220 to take a closer look, or to capture an image. In this regard, the patient, or a local person working with the patient, can press the button 242 and freeze a frame of the camera 220, in accordance with various embodiments.

Similarly, the button 244 can be configured to freeze a frame of the camera 230 for similar reasons, in accordance with various embodiments.

In various embodiments, the portable medical device 200 further comprises a plurality of lights 252 disposed circumferentially around the camera 220. In various embodiments, each light in the plurality of lights 252 can comprise a light emitting diode (“LED”), an incandescent light, a compact fluorescent light (“CFL”), a halogen light, or the like. The present disclosure is not limited in this regard. In various embodiments, each light in the plurality of lights 252 comprises an LED. In various embodiments, the LED can be configured to emit an electromagnetic radiation with an average wavelength between 780 nm and 1 mm (i.e., an infrared LED). In various embodiments, each light in the plurality of lights 252 includes a color temperature between 3700K and 4700K, or approximately 4300K. In this regard, the plurality of lights 252 can be configured to emit a bright white color to illuminate an object being inspected by the portable medical device 200.

In various embodiments, the portable medical device 200 further comprises a telemedicine management system 300. For example, the electronic system 260 can comprise a printed circuit board disposed within the main body 210 of the portable medical device 200. In this regard, the printed circuit board can include various electrical components of the portable medical device 200, in accordance with various embodiments.

In various embodiments, the electronic system 260 is configured to facilitate communications between the portable medical device 200 and a telemedicine management system 300 of a telemedicine system. In this regard, the electronic system 260 is configured to transmit video data from cameras 220, 230, in accordance with various embodiments. In various embodiments, the telemedicine management system 300 can be capable of controlling various aspects of the portable medical device 200 (e.g., freezing a frame of a camera, zooming in or out with the camera, or the like). The present disclosure is not limited in this regard.

Referring now to FIG. 2C, an exploded view of a portable medical kit 299 is illustrated, in accordance with various embodiments. The portable medical kit 299 includes the portable medical device 200 and detachable components (e.g., a tongue depressor apparatus 270 and an extended mirror). In this regard, the portable medical device 200 can be adaptable by coupling a detachable component to the main body 210 of the portable medical device 200. For example, the tongue depressor apparatus 270 is configured to couple to the main body 210 of the portable medical device 200 as discussed in more detail herein.

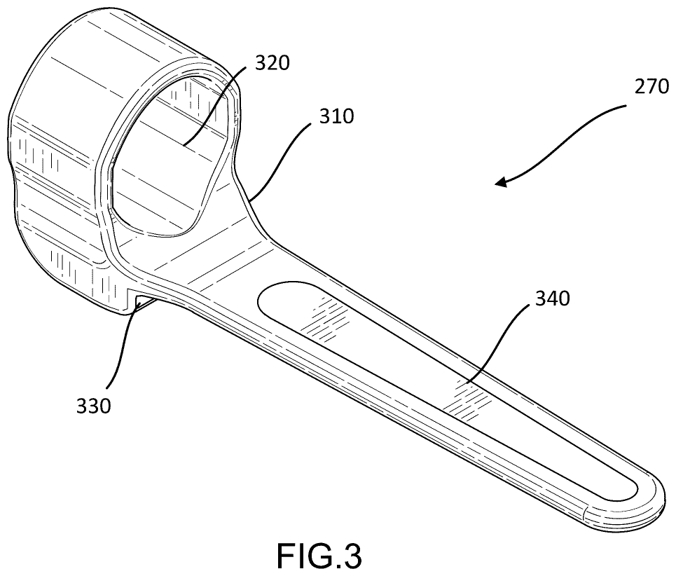

Referring now to FIG. 3, a top perspective view of a tongue depressor apparatus 270 for facilitating a remote diagnosis of a patient is illustrated, in accordance with various embodiments. In various embodiments, as described further herein, the tongue depressor apparatus 270 is configured to be coupled to a portable medical device 200. In this regard, when using the tongue depressor apparatus 270 with a portable medical device 200, the portable medical device 200 can be configured to facilitate a telemedicine visit (e.g., by a parent, sibling, guardian, the patient, etc.). Stated another way, the tongue depressor apparatus 270 can be utilized with a portable medical device 200 to facilitate a throat examination in a remote location from a doctor's office. In various embodiments, the tongue depressor apparatus 270 can be configured to facilitate communications with a doctor in an emergency situation (e.g., on an airplane, a boat, or the like). The present disclosure is not limited in this regard.

As shown in FIG. 3, the tongue depressor apparatus 270 comprises a tongue depressor apparatus main body 310 extending longitudinally from a first end to a second end, and an aperture 320 disposed through the tongue depressor apparatus main body 310 from the first end to the second end, the aperture 320 defining a longitudinal axis. In various embodiments, the aperture 320 of the tongue depressor apparatus 270 can be configured to be detachably coupled with a portable medical device (e.g., the portable medical device 200 from FIG. 3 as described further herein) for virtual medical visits.

In various embodiments, the tongue depressor apparatus 270 comprises a flange 340 extending axially from the second end of the tongue depressor apparatus main body 310 and away from the first end of the tongue depressor apparatus main body 310. In an example embodiment, the flange 340 has a length of at least three times the width of the flange 340. However, the present disclosure is not limited in this regard. In other example embodiments, the flange 340 may have a length that is at least two times the width of the flange 340. In various example embodiments, the flange 340 may contain a radiused distal end.

The tongue depressor apparatus 270 comprises a slot 330 extending from the first end to the second end, the slot 330 disposed radially outward from the flange 340 relative to the longitudinal axis, the slot 330 defining a centerline that is parallel to the longitudinal axis. In example embodiments, the slot 330 of the tongue depressor apparatus 270 is configured to hold and retain a disposable tongue depressor 550. For example, the slot 330 can be sized and configured to generate an interference fit with a disposable tongue depressor 550. Stated another way, in response to inserting the disposable tongue depressor 550 into the slot, a friction force can be generated by an internal surface of the slot 330. Once the disposable tongue depressor 550 is slid to a desired position, the friction generated from sliding the disposable tongue depressor 550 axially within the slot can retain the disposable tongue depressor 550 during operation of the oral examination system described further herein. The disposable tongue depressor 550 could be similar to any disposable tongue depressor 550 that is available off the shelf (i.e., found at retailers). The slot 330 being configured to work with a wide variety of disposable tongue depressors 550 in both wood and polymer based materials.

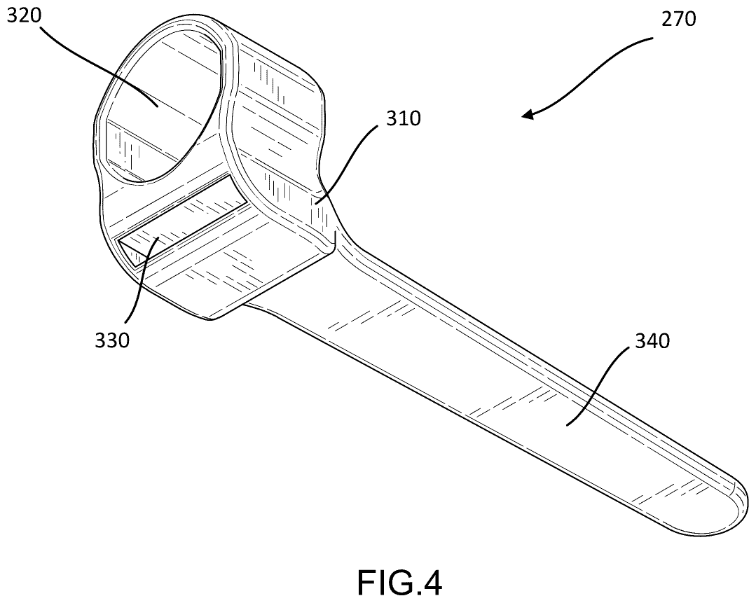

Referring now to FIG. 4, a bottom perspective view of a tongue depressor apparatus 270. This figure in its alternate view to better show that the aperture 320 and the slot 330 extending from the first end of the tongue depressor apparatus main body 310 through to the second end of the tongue depressor apparatus main body 310.

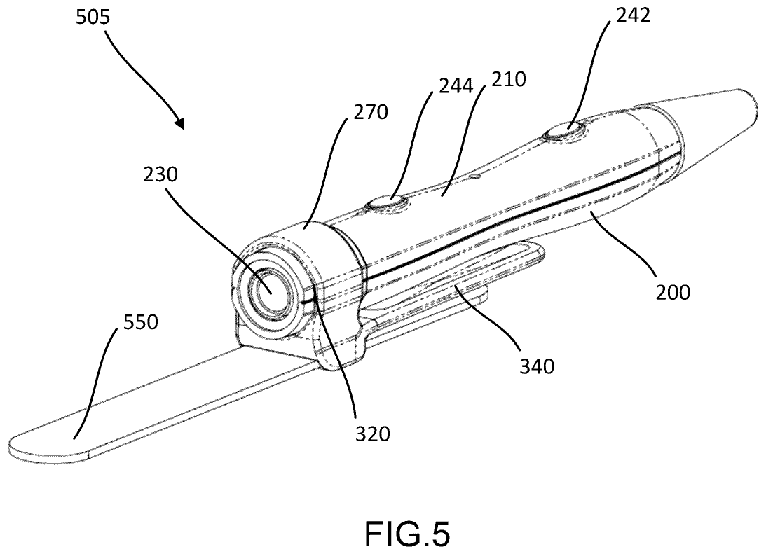

Referring now to FIG. 5, a perspective view of an oral examination camera system 505 for facilitating a remote diagnosis of a patient is illustrated, in accordance with various embodiments. In example embodiments, the oral examination camera system 505 can be configured to have a tongue depressor apparatus 270 detachably coupled to a portable medical device 200. In example embodiments, the oral examination camera system 505 can also be configured to hold a disposable tongue depressor 550 inserted into the tongue depressor apparatus 270. In other example embodiments, when the tongue depressor apparatus 270 is detachably coupled to the portable medical device 200, the flange 340 allows a user to clip the oral examination camera system 505 to their clothing (e.g., a shirt pocket, a belt, or the like) for ease of use and safe keeping.

In example embodiments, the portable medical device 200 can be configured to have a camera 230 at the first end. The camera 230 is shaped in a way that allows the aperture 320 of the tongue depressor apparatus 270 to surround the camera 230 and thus allowing the tongue depressor apparatus 270 to detachably couple to the portable medical device 200. In various example embodiments, when the oral examination camera system 505 has been configured with the tongue depressor apparatus 270, detachably coupled to the portable medical device 200, and a disposable tongue depressor 550 has been inserted into the tongue depressor apparatus 270, a user can then use the oral examination camera system 505 to conduct an oral examination of a patient. In various embodiments, to assist in an examination, a user can hold the oral examination camera system 505 by the main body 210 of the portable medical device 200. Additionally, while a user is depressing the tongue of a patient, the user may record the examination after a button 244 controlling a parameter is pressed that is coupled to control the recording feature of the camera 230. The button 244 being located adjacent to the main body 210 on the portable medical device 200. In various embodiments, the camera 230 can be electronically coupled to the button 244 so that the recording can be wirelessly transmitted to a user device for review and diagnosis. The user device could be found locally in the same room as the oral examination camera system 505, or the user device could be in a remote location.

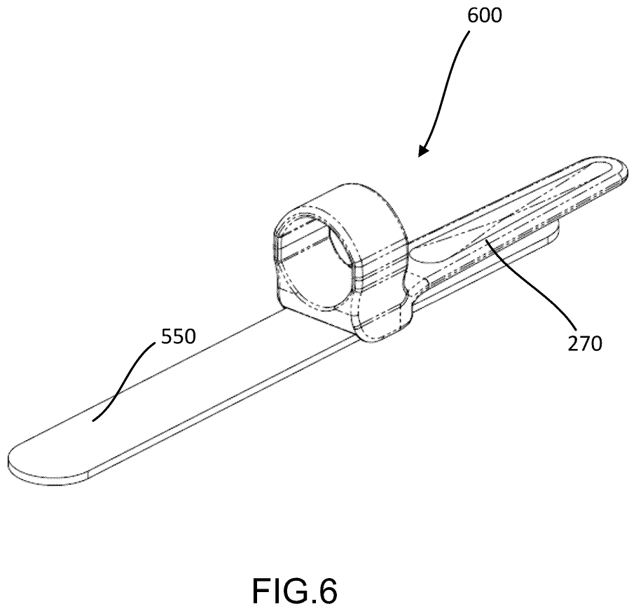

Referring now to FIG. 6, a perspective view of a tongue depressing system 600 which can contain a tongue depressor apparatus 270 and a disposable tongue depressor 550. In various embodiments, the tongue depressor apparatus 270 can be configured to allow the slot 330 to work with a wide variety of disposable tongue depressors 550 in both wood and polymer based materials. In example embodiments, a user can remove the tongue depressor apparatus 270 from the portable medical device 200 so that the user can then insert a disposable tongue depressor 550. Some users may find the tongue depressing system 600 easier to maneuver while inserting a disposable tongue depressor 550. In other example embodiments, the user can also remove the tongue depressing system 600 from the portable medical device 200 when the user feels that the tongue depressor apparatus 270 may have encountered a patient's mouth or patient's saliva directly.

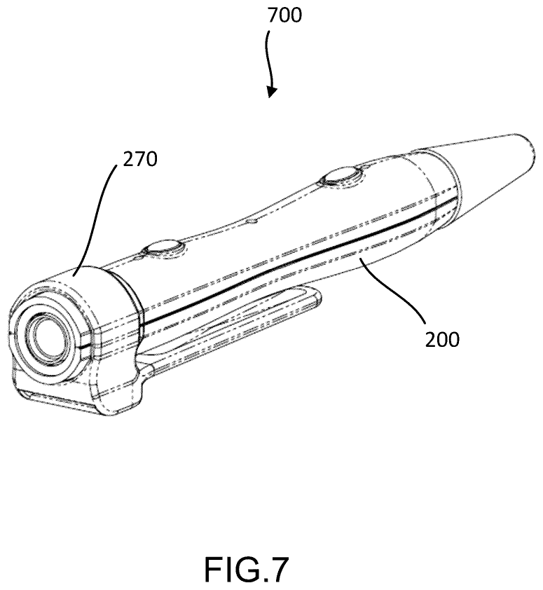

Referring now to FIG. 7, a perspective view of a portable medical system 700 which can contain a tongue depressor apparatus 270 and a portable medical device 200. In various embodiments, the portable medical system 700 can be the configuration that a user could store the tongue depressor apparatus 270 while it is detachably coupled to the portable medical device 200. With the tongue depressor apparatus 270 detachably coupled to the portable medical device 200, the flange 340 then can be configured to be used as a clip, allowing a user to hang the portable medical system 700 from their clothing for safe keeping while not in use. When a user is prepared to give an examination, the user can then remove the portable medical system 700 from their clothing, insert a disposable tongue depressor 550 into the slot 330 of the tongue depressor apparatus 270, and then conduct the examination of the patient. In various embodiments, the user could then remove the disposable tongue depressor 550 from the slot 330 of the tongue depressor apparatus 270 and then attach the portable medical system 700 to the user's clothing by way of the flange 340.

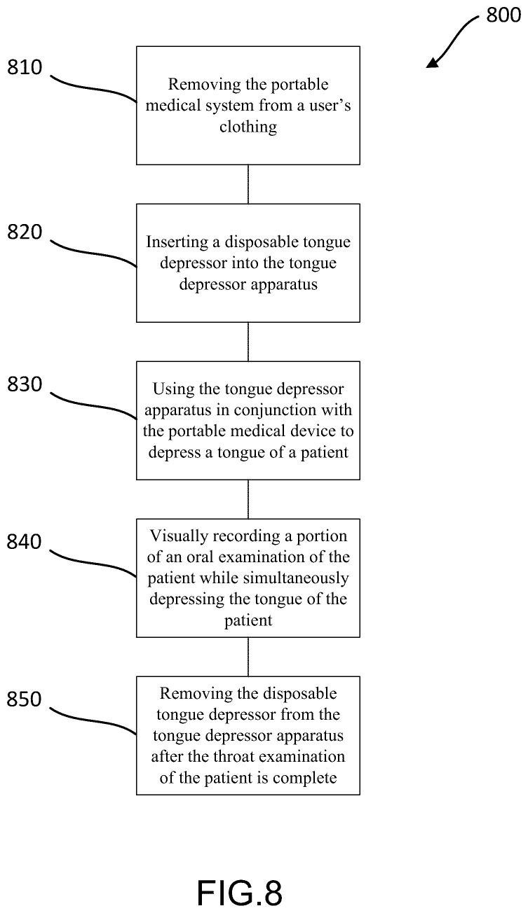

Referring now to FIG. 8, which illustrates a method 800 for using an oral examination camera system 505 to conduct an oral examination on a patient. Method 800 may first include a user removing the portable medical system 700 from the user's clothing (operation 810). The method 800 can then include inserting a disposable tongue depressor 550 into the tongue depressor apparatus 270 (operation 820). The method 800 can then include using the tongue depressor apparatus 270 in conjunction with the disposable tongue depressor 550 and portable medical device 200 to depress the tongue of a patient (operation 830). The method 800 can include a user visually recording during a portion of an oral examination of a patient while simultaneously depressing the tongue of the patient by pressing the button 244 on the portable medical device 200 (Operation 840). The method 800 can include removing the disposable tongue depressor 550 from the tongue depressor apparatus 270 after the oral examination of the patient has been completed (Operation 850).

The process flows depicted are merely embodiments and are not intended to limit the scope of the disclosure. For example, the steps recited in any of the method or process descriptions may be executed in any order and are not limited to the order presented. It will be appreciated that the following description makes appropriate references not only to the steps and user interface (UI) elements, but also to the various system components as described herein. It should be understood that, although exemplary embodiments are illustrated in the figures and described herein, the principles of the present disclosure may be implemented using any number of techniques, whether currently known or not. The present disclosure should in no way be limited to the exemplary implementations and techniques illustrated in the drawings and described below. Unless otherwise specifically noted, articles depicted in the drawings are not necessarily drawn to scale.

Benefits, other advantages, and solutions to problems have been described herein regarding specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B, and C may be present in a single embodiment; for example, A and B, A and C, B and C, or A and B and C. Different cross-hatching is used throughout the figures to denote different parts but not necessarily to denote the same or different materials.

Systems, methods, and apparatus' are provided herein. In the detailed description herein, references to “one embodiment,”“an embodiment,”“various embodiments,” etc., indicate that the embodiment described may include a particular feature, structure, or characteristic, but every embodiment may not necessarily include the feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 212 (f) unless the element is expressly recited using the phrase “means for.” As used herein, the terms “comprises,”“comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Finally, any of the above-described concepts can be used alone or in combination with any or all the other above-described concepts. Although various embodiments have been disclosed and described, one of ordinary skill in this art would recognize that certain modifications would come within the scope of this disclosure. Accordingly, the description is not intended to be exhaustive or to limit the principles described or illustrated herein to any precise form. Many modifications and variations are possible considering the above teaching.

What is claimed is:

1. An oral examination camera system, the oral examination camera system comprising:
a portable medical device including a portable medical device main body, a camera, and a button, the portable medical device main body extending from a first end to a second end, the camera disposed at the first end, the button configured to control a parameter of the camera; and
a tongue depressor apparatus configured to removably couple to the portable medical device, the tongue depressor apparatus including a main body extending from the first end to the second end, an aperture disposed through the main body from the first end to the second end, the aperture defining a longitudinal axis, a flange extending axially from the second end of the main body and away from the first end of the main body, and a slot extending from the first end to the second end, the slot disposed radially outward from the flange relative to the longitudinal axis, the slot defining a centerline that is parallel to the longitudinal axis.

2. The oral examination camera system of claim 1, wherein the slot is configured to allow a disposable tongue depressor to be inserted into the tongue depressor apparatus.

3. The oral examination camera system of claim 2, wherein the disposable tongue depressor can comprise wood or polymer based disposable tongue depressors.

4. The oral examination camera system of claim 3, wherein the aperture is configured to detachably couple the tongue depressor apparatus to the portable medical device.

5. The oral examination camera system of claim 4, wherein the aperture is configured to detachably couple the tongue depressor apparatus to the portable medical device by surrounding the camera.

6. The oral examination camera system of claim 5, wherein the tongue depressor apparatus is configured where the flange is orientated in a direction from the first end of the portable medical device to the second end.

7. The oral examination camera system of claim 6, wherein the flange is configured to allow a user to clip the oral examination camera system to an article of clothing of a user.

8. The oral examination camera system of claim 7, wherein the button is configured to control the recording of the camera after being pressed by the user.

9. The oral examination camera system of claim 8, wherein the recording of the camera is configured to be wirelessly transmitted on a user device.

10. The oral examination camera system of claim 9, wherein the user device could be locally in the same room as the oral examination camera system or in a remote location.

11. A tongue depressor apparatus, comprising:
a main body extending from a first end to a second end;
an aperture disposed through the main body from the first end to the second end, the aperture defining a longitudinal axis;
a flange extending axially from the second end of the main body and away from the first end of the main body; and
a slot extending from the first end to the second end, the slot disposed radially outward from the flange relative to the longitudinal axis, the slot defining a centerline that is parallel to the longitudinal axis.

12. The tongue depressor apparatus of claim 11, wherein the aperture is configured to detachably couple to a portable medical device adjacent to a handle.

13. The tongue depressor apparatus of claim 12, wherein the flange has a length of at least three times a width of the flange.

14. The tongue depressor apparatus of claim 13, wherein the flange has a radiused distal end.

15. The tongue depressor apparatus of claim 14, where the flange is configured to allow a user to clip the portable medical device to an article of clothing of a user when the tongue depressor apparatus is detachably coupled to the portable medical device.

16. The tongue depressor apparatus of claim 15, wherein the slot is configured to hold a disposable tongue depressor during an oral examination.

17. The tongue depressor apparatus of claim 16, wherein the disposable tongue depressor can comprise wood or polymer based disposable tongue depressors.

18. A method for using an oral examination camera system, the method comprising:
removing a portable medical system from a user's clothing;
inserting a disposable tongue depressor into a tongue depressor apparatus;
coupling the tongue depressor apparatus to a portable medical device;
using the tongue depressor apparatus in conjunction with the disposable tongue depressor and the portable medical device to depress a tongue of a patient and visually record a portion of an oral examination of the patient simultaneously with the depressing of the tongue of the patient; and
removing the disposable tongue depressor from the tongue depressor apparatus after the oral examination of the patient is complete.

19. The method for using an oral examination camera system of claim 18, wherein the disposable tongue depressor is wood or polymer based.

20. The method for using an oral examination camera system of claim 19, wherein the recording of the oral examination can be wirelessly transmitted to a user device.
